(19) United States
(12) Patent Application Publication
(10) Pub. No.: US 2024/0272256 A1
(43) Pub. Date: Aug. 15, 2024

(54) LIGHT GAUSS GUARD FOR PORTABLE MAGNETIC RESONANCE IMAGING DEVICES

(71) Applicant: Hyperfine Operations, Inc., Guilford, CT (US)

(72) Inventors: Michael Stephen Poole, Guilford, CT (US); Michael Twieg, New Haven, CT (US); Anne Michele Nelson, Guilford, CT (US); Richard Q. Wang, New Haven, CT (US)

(73) Assignee: Hyperfine Operations, Inc., Guilford, CT (US)

(21) Appl. No.: 18/593,436

(22) Filed: Mar. 1, 2024

Related U.S. Application Data
(63) Continuation of application No. PCT/US22/42419, filed on Sep. 2, 2022.
(60) Provisional application No. 63/240,252, filed on Sep. 2, 2021.

(51) Int. Cl. G01R 33/44; G01R 33/38

(52) U.S. Cl. CPC G01R 33/445; G01R 33/3802; G01R 33/3806

(57) ABSTRACT

According to some aspects, a device configured to be coupled to a portable magnetic resonance imaging (MRI) device is provided, the device comprising at least one light source arranged to, when operated, project a visible boundary around at least a portion of the portable MRI device, wherein the visible boundary demarcates a region within which a magnetic field strength of a magnetic field generated by the portable MRI device equals or exceeds a threshold. The at least one light source may be arranged such that an angle of the at least one light source relative to the portable MRI device is adjustable and wherein adjusting the angle of the at least one light source relative to the portable MRI device changes a shape and/or size of the visible boundary.

### CROSS REFERENCE TO RELATED APPLICATIONS

This application claims the benefit of priority under 35 U.S.C. § 120 as a continuation of PCT Patent Application No. PCT/US2022/042419, filed on Sep. 2, 2022, which claims the benefit of priority to U.S. provisional patent application Ser. No. 63/240,252, filed on Sep. 2, 2021, and titled “LIGHT GAUSS GUARD FOR PORTABLE MAGNETIC RESONANCE IMAGING DEVICES,” which are incorporated by reference herein in their entirety.

### BACKGROUND

MRI provides an important imaging modality for numerous applications and is widely utilized in clinical and research settings to produce images of the inside of the human body. As a generality, MRI is based on detecting magnetic resonance (MR) signals, which are electromagnetic waves emitted by atoms in response to state changes resulting from applied electromagnetic fields. For example, nuclear magnetic resonance (NMR) techniques involve detecting MR signals emitted from the nuclei of excited atoms upon the re-alignment or relaxation of the nuclear spin of atoms in an object being imaged (e.g., atoms in the tissue of the human body). Detected MR signals may be processed to produce images, which in the context of medical applications, allows for the investigation of internal structures and/or biological processes within the body for diagnostic, therapeutic and/or research purposes.

### SUMMARY

Some embodiments provide for a device configured to be coupled to a portable magnetic resonance imaging (MRI) device, the device comprising: at least one light source arranged to, when operated, project a visible boundary around at least a portion of the portable MRI device, wherein the visible boundary demarcates a region within which a magnetic field strength of a magnetic field generated by the portable MRI device equals or exceeds a threshold.

Some embodiments provide for a system, comprising: a portable magnetic resonance imaging (MRI) device; and a device coupled to the portable MRI device comprising at least one light source arranged to, when operated, project a visible boundary around at least a portion of the portable MRI device, wherein the visible boundary demarcates a region within which a magnetic field strength of a magnetic field generated by the portable MRI device equals or exceeds a threshold.

Some embodiments provide for a method for operating a magnetic resonance imaging (MRI) device, the MRI device being coupled to a device comprising at least one light source arranged to, when operated, project a visible boundary around at least a portion of the portable MRI device, wherein the visible boundary demarcates a region within which a magnetic field strength of a magnetic field generated by the portable MRI device equals or exceeds a threshold, the method comprising: operating the device to project the visible boundary; prior to imaging, using the MRI device, operating the device to stop projecting the visible boundary; and imaging, using the MRI device.

### BRIEF DESCRIPTION OF THE DRAWINGS

Various aspects and embodiments of the application will be described with reference to the following figures. It should be appreciated that the figures are not necessarily drawn to scale. Items appearing in multiple figures are indicated by the same reference number in all the figures in which they appear.

FIG. 1 is an exemplary portable medical imaging device for use in accordance with some embodiments of the technology described herein.

FIG. 2A, 2B and 2C are top, front and side views of a portable medical imaging device, for example of the portable medical imaging device shown in FIG. 1, illustrating example magnetic fringe fields associated with the device.

FIG. 3A illustrates the portable medical imaging device of FIG. 1 with a device configured to project a visible boundary around at least a portion of the portable medical imaging device, in accordance with some embodiments of the technology described herein.

FIG. 3B illustrates the portable medical imaging device of FIG. 1 with a device configured to project a visible boundary around at least a portion of the portable medical imaging device as well as the magnetic field generated by the portable medical imaging device, in accordance with some embodiments of the technology described herein.

FIG. 4 illustrates another view of the device of FIG. 3A, in accordance with some embodiments of the technology described herein.

FIG. 5A illustrates an example optical module for device of FIG. 3A, in accordance with some embodiments of the technology described herein.

FIGS. 5B-5E illustrate example components of an optical module for the device of FIG. 3A, in accordance with some embodiments of the technology described herein.

FIGS. 6A-6B illustrate another example optical module for the device of FIG. 3A, in accordance with some embodiments of the technology described herein.

FIG. 7 illustrates an example process for using a light Gauss guard with a portable medical imaging device, in accordance with some embodiments of the technology described herein.

### DETAILED DESCRIPTION

### Introduction

The present disclosure relates generally to magnetic resonance imaging (MRI) devices and, more specifically, a device comprising at least one light source arranged to project a visible boundary that surrounds at least a portion of the MRI device. The visible boundary may demarcate a region within which the magnetic field generated by the MRI device equals or exceeds a threshold. For example, the visible boundary may, in some embodiments, indicate a 5-Gauss line for the portable MRI device.

The MRI scanner market is overwhelmingly dominated by high-field systems, with the vast majority of clinical MRI scanners operating at 1.5 T or 3 T, with higher field strengths of 7 T and 9 T used in research settings. As used herein, “high-field” refers generally to MRI systems presently in use in a clinical setting and, more particularly, to MRI systems operating with a main magnetic field (i.e., a B₀ field) at or above 1.5 T, though clinical systems operating between 0.5 T and 1.5 T are often also characterized as “high-field.” Field strengths between 0.2 T and 0.5 T have been characterized as “mid-field” and, as field strengths in the high-field regime have continued to increase, field strengths in the range between 0.5 T and 1 T have also been characterized as mid-field. By contrast, “low-field” refers to MRI systems operating with a B₀ field of less than or equal to 0.2 T, though systems having a B₀ field of between 0.2 T and 0.3 T have sometimes been characterized as low-field as a consequence of increased field strengths at the high end of the high-field regime. Within the low-field regime, low-field MRI systems operating with a B₀ field of less than 0.1 T are referred to herein as “very low-field” and low-field MRI systems operating with a B₀ field of less than 10 milliTesla (mT) are referred to herein as “ultra-low field”.

More recently, certain advancements (such as those developed by the assignee of the instant application) have paved the way for improved quality, portable and/or lower-cost low-field MRI systems that can, in turn, drive wide-scale deployability of MRI technology in a variety of environments beyond the large MRI installments at hospitals and research facilities. As such, low-field MRI presents an attractive imaging solution, providing a relatively low cost, high availability alternative to high-field MRI. In particular, low-field MRI systems can be implemented as self-contained systems that are deployable in a wide variety of clinical settings where high-field MRI systems cannot, for example, by virtue of being transportable, cartable or otherwise generally mobile so as to be deployable where needed. As a result of this portability, such low-field MRI systems may be expected to operate in generally unshielded or partially shielded environments (e.g., outside of specially shielded rooms or encompassing cages) while also handling the particular noise environment in which they are deployed.

The inventors have recognized that with the emergence of a new paradigm for MRI, certain additional challenges may arise with respect to a portable, point-of-care (POC) MRI system that can be installed in a variety of settings such as an emergency room, office or clinic. For example, when in storage or when transported from location to location, a portable, low-field POC MRI system (including any of the systems described herein) may temporarily reside in (or pass through) an area or areas that are not access controlled. On the one hand, a low-field system MRI system operates at a static magnetic field much lower than that of conventional high-field MRI systems, and as such certain risks typically associated with high-field systems (e.g., potential projectile effects) are likely absent. On the other hand, there still may be other concerns associated with having even low-level static magnetic fields present in areas that are not access controlled. Examples of such concerns may include, but are not necessarily limited to: individuals having active implants (e.g., pacemakers, defibrillators, insulin pumps, deep brain stimulators, vagus nerve stimulators, cochlear implants, etc.) in the vicinity of the MRI system; individuals with metal containing tattoos or permanent make-up on the head or neck regions in the vicinity of the MRI system; and individuals with suspected metal present in the eye (e.g., metal workers, injury victim, etc.) in the vicinity of the MRI system.

High fringe fields may be dangerous to bystanders for the reasons discussed herein, however low-strength fringe fields (e.g., fringe fields having a strength of less than 30 Gauss, less than 25 Gauss, less than 20 Gauss, less than 15 Gauss, less than 10 Gauss, less than 5 Gauss, less than 2 Gauss, less than 1 Gauss, any strength in the range of 2-10 Gauss or 2-20 Gauss, etc.) may be tolerated because such low-strength fringe fields may not present a safety concern or otherwise interfere with operation of nearby electronics including implants (e.g., pacemakers) or other electronic devices (e.g., medical instruments, smartphones, etc.).

In some environments, safety regulations may require indications of the boundary or perimeter within which the magnetic field of the MRI system exceeds a given threshold field strength. These boundaries are sometimes called “Gauss lines.” A Gauss line for a device may indicate a region, outside of which, the strength of a magnetic field generated by the device is less than a threshold strength. For example, the 5 Gauss line for an MRI device may indicate a region outside of which the magnetic field generated by the MRI device has a strength of less than 5 Gauss. Magnetic fields having strength higher than 30 Gauss may present projectile hazards. Some safety regulations may require the 5, 10 and 200 Gauss lines to be indicated to demarcate the physical perimeters within which the respective thresholds are exceeded.

It should be appreciated that such challenges are generally not of concern with respect to the more conventional, high field MRI systems that are typically immobile and installed in specialized rooms with extensive shielding and defined access control protocols. For example, compliance with the above-mentioned safety regulations may be achieved by indicating the 5, 10 and 200 Gauss lines on the floor of the room in which the MRI system is installed, to remind personnel where the respective protocols need to be enforced. This solution is generally inapplicable in the context of portable MRI systems because the perimeters requiring demarcation would need to move along with the MRI device. In view of this and as described herein, embodiments of the disclosure provide for a device configured to be coupled to a portable medical imaging device that demarcates a region within which a magnetic field strength of the MRI device exceeds a threshold. When operated, the device projects a visible boundary around at least a portion of the MRI device which inhibits encroachment within a region inside the boundary.

The inventors have recognized that the inclusion of such a device which inhibits encroachment of the MRI device is particularly important in embodiments in which the portable medical imaging device includes one or more permanent magnets. Unlike other magnetic assemblies, a magnetics component comprising a permanent magnet produces fringe fields both during operation of the medical imaging device and during transport and storage of the medical imaging device when the portable medical imaging device is otherwise not being operated. As described herein, transport and storage of the portable medical imaging device may involve the device entering uncontrolled areas where bystanders may be present, such as a hallway. Thus, when a portable medical imaging device includes one or more permanent magnets (e.g., to generate the B₀ field), it is important to provide an indication of a boundary demarcating the region in which it is unsafe for bystanders or electronic devices to enter due to fringe fields produced during operation, transport, and storage of the portable medical imaging device.

In some embodiments, the device may be configured to project a visible boundary corresponding to a particular Gauss line. For example, in some embodiments, the device may project a visible boundary such that the region within the visible boundary includes a particular Gauss line (e.g., the 5 Gauss line, the 10 Gauss line, etc.).

The inventors have recognized that devices of the type described herein which are capable of projecting, with at least one light source, a visible boundary around at least a portion of the MRI device are advantageous for a number of reasons. For example, with devices of the type described herein, the visible boundary may be automatically deployed electronically, without the need to manually position parts of the device. In some embodiments, the device may be configured to automatically project the boundary in response to an indication that the MRI device is or is about to perform imaging. In some embodiments, the device may be configured to automatically project the boundary in response to movement of the MRI device.

In addition, the device described herein provides less operator interference than mechanical devices, for example. In particular, the visible boundary projected by the device provides an indication of a magnetic field strength of the MRI device without physically inhibiting operators who may need to access the MRI device to operate the MRI device.

Further still, in some embodiments, the device may be easily adjustable as needs for the MRI device change. For example, in some embodiments, it may be desired to store the MRI device against a wall while still inhibiting encroachment on the MRI device from a side of the MRI device that is accessible (e.g., a portion of the MRI device that does not face the wall). The device may be easily adjusted to project a visible boundary that surrounds only a portion of the MRI device (e.g., by powering only certain ones of the device light sources). In addition, a size and/or shape of the visible boundary projected by the device may be easily adjusted as necessary (e.g., where the magnetic field generated by the MRI device changes) by adjusting angles of one or more light sources of the device relative to the MRI device.

Accordingly, the inventors have developed a device configured to project a visible boundary around at least a portion of the MRI device.

Some embodiments provide for a device configured to be coupled to a portable magnetic resonance imaging (MRI) device, the device comprising: at least one light source (e.g., at least one light emitting diode) arranged to, when operated, project a visible boundary around at least a portion of the portable MRI device, wherein the visible boundary demarcates a region within which a magnetic field strength of a magnetic field generated by the portable MRI device equals or exceeds a threshold.

In some embodiments, the at least one light source may be arranged to, when operated, project a visible boundary that surrounds the portable MRI device. In some embodiments, the at least one light source may be arranged to, when operated, project a visible boundary that is radially symmetrical. In some embodiments, the at least one light source may be arranged to, when operated, project a visible boundary that is asymmetrical.

In some embodiments, the at least one light source comprises a plurality of light sources (e.g., at least 10 light sources, at least 20 light sources).

In some embodiments, the at least one light source comprises a first light source, wherein an angle of the first light source relative to the portable MRI device may be adjustable such that adjusting the angle of the first light source changes a shape and/or size of the visible boundary. In some embodiments, the at least one light source comprises multiple light sources, and wherein respective angles of the multiple light sources may be adjusted independently of one another such that different light sources of the multiple light sources may be positioned at different angles relative to the portable MRI device. In some embodiments, the device may be further configured to hold the first light source at a fixed angle relative to the portable MRI device.

In some embodiments, the at least one light source may be arranged to, when operated, project a visible boundary comprising text. In some embodiments, the text comprises an indication of the magnetic field strength at or within the visible boundary.

In some embodiments a brightness of the at least one light source may be set based on a brightness of ambient lighting in an environment of the portable MRI device.

In some embodiments, the magnetic field strength within the region may be between 1 Gauss and 30 Gauss. In some embodiments, the magnetic field strength within the region may be between 1 Gauss and 10 Gauss. In some embodiments, the visible boundary indicates a 5 Gauss line of the portable MRI device.

In some embodiments, the at least one light source may be arranged to, when operated, project a plurality of projections. The plurality of projections may be spaced equidistantly from each other. In some embodiments, the at least one light source may be arranged to, when operated, project a continuous projection of light.

In some embodiments, the device further comprises a housing for the at least one light source. In some embodiments, the device further comprises at least one optical module comprising the at least one light source and a circuit board. In some embodiments, each optical module further comprises one or more lenses.

In some embodiments, the at least one light source may be arranged to, when operated, alternate between projecting the visible boundary and not projecting the visible boundary. In some embodiments, the at least one light source alternates between projecting the visible boundary and not projecting the visible boundary at a predefined frequency.

Some embodiments provide for a system, comprising: a portable magnetic resonance imaging (MRI) device; and a device coupled to the portable MRI device comprising at least one light source arranged to, when operated, project a visible boundary around at least a portion of the portable MRI device, wherein the visible boundary demarcates a region within which a magnetic field strength of a magnetic field generated by the portable MRI device equals or exceeds a threshold. The device may be configured according to the aspects described herein.

In some embodiments, the device may be coupled to the portable MRI device below an imaging region of the portable MRI device. In some embodiments, the device may be coupled to the portable MRI device above an imaging region of the MRI device. In some embodiments, the portable MRI device further comprises a base, the base supporting a magnetics system of the portable MRI device and housing a power system for the portable MRI device, wherein the base further comprises at least one conveyance mechanism allowing the portable MRI device to be transported to different locations, and the device may be coupled to the base of the portable MRI device.

In some embodiments, the at least one light source may be arranged to, when operated, project the visible boundary onto a surface which supports the portable MRI device (e.g., a ramp, a vehicle bed, a floor).

Some embodiments provide for a method for operating a magnetic resonance imaging (MRI) device, the MRI device being coupled to a device comprising at least one light source arranged to, when operated, project a visible boundary around at least a portion of the portable MRI device, wherein the visible boundary demarcates a region within which a magnetic field strength of a magnetic field generated by the portable MRI device equals or exceeds a threshold, the method comprising: operating the device to project the visible boundary; prior to imaging, using the MRI device, operating the device to stop projecting the visible boundary; and imaging, using the MRI device.

In some embodiments, the method further comprises transporting the MRI device to a second location while operating the device.

In some embodiments, operating the device to project the visible boundary comprises powering the at least one light source so that the at least one light source projects the visible boundary around at least the portion of the MRI device. In some embodiments, operating the device to project the visible boundary comprises operating the at least one light source to alternate between projecting the visible boundary and not projecting the visible boundary.

The aspects and embodiments described above, as well as additional aspects and embodiments, are described further below. These aspects and/or embodiments may be used individually, all together, or in any combination, as the technology is not limited in this respect.

For ease of explanation, embodiments of a deployable guard device disclosed herein are described in the context of a portable POC MRI system; however, it should be appreciated that such a guard device may also be used in conjunction with other devices including, but not limited to, X-ray images, CT imaging devices, etc.

### Example Devices

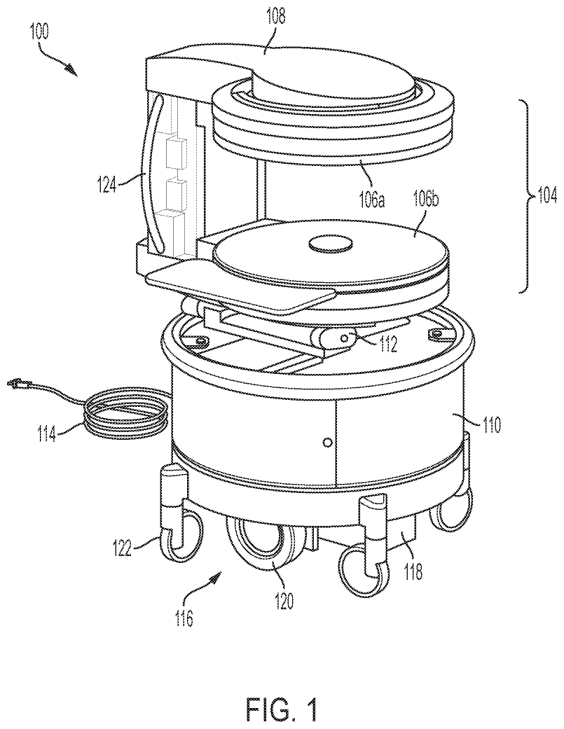

Referring initially to FIG. 1, there is shown an exemplary portable medical imaging device 100 (also referred to herein as a portable MRI device) for use in accordance with embodiments of the technology described herein. In the embodiment depicted in FIG. 1, the portable medical imaging device 100 may be a POC MRI system including a B₀ magnet 104 having at least one first permanent magnet 106a and at least one second permanent magnet 106b magnetically coupled to one another by a ferromagnetic yoke 108 configured to capture and channel magnetic flux to increase the magnetic flux density within the imaging region (field of view) of the MRI device 100. In some embodiments, the first permanent magnet 106a and second permanent magnet 106b each comprises a plurality of concentric permanent magnet rings. Alternatively, in some embodiments, B₀ magnet 104 may be formed using electromagnets, laminate magnets, or hybrid magnets. Additional information regarding the formation of B₀ magnet 104 may be found in U.S. Patent Publication No. US 2018/0143274, filed Nov. 22, 2017, and titled “Low-Field Magnetic Resonance Imaging Methods and Apparatus”, hereby incorporated by reference in its entirety.

In some embodiments, the B₀magnet 104 may be configured to generate a B₀ field having a strength in the low-field regime—between 0.1 T and 0.2 T. For example, the strength of B₀ field of the portable MRI device 100 may be in the range of 0.05 T and 0.1 T.

In some embodiments, the B₀ magnet 104 may be coupled to or otherwise attached or mounted to a base 110 by a positioning mechanism 112 (such as for example a goniometric stage) so that the B₀ magnet can be tilted (e.g., rotated about its center of mass) to provide an incline to accommodate a patient's anatomy as needed. In addition to providing a load bearing structure(s) for supporting the B₀ magnet 104, the base 110 may also include an interior space or compartment(s) configured to house the electronics (not shown) used to operate the portable MRI device 100. For example, the base 110 may house power components to operate gradient coils (e.g., X, Y and Z) and RF transmit/receive coils, as well as RF coil amplifiers (power amplifiers to operate the transmit/receive coils of the system), power supplies, console, power distribution unit and other electronics needed to operate the MRI device.

In some embodiments, the electronics needed to operate portable MRI device 100 may consume less than 1 kW of power and, in some embodiments, less than 750 W of power (e.g., MRI systems utilizing a permanent B₀ magnet solution). However, devices that consume greater power may also be utilized as well, as the aspects of the technology described herein are not limited in this respect. As such, the exemplary portable MRI device 100 may be powered via a single power connection 114 configured to connect to a source of mains electricity, such as an outlet providing single-phase power (e.g., a standard or large appliance outlet). Accordingly, the portable MRI device 100 can be plugged into a single available power outlet and operated therefrom. Aspects of power systems that may be used as part of portable MRI device 100 are described in U.S. Patent Publication No. US 2018/0143274, filed Nov. 22, 2017, and titled “Low-Field Magnetic Resonance Imaging Methods and Apparatus”, which is incorporated by reference herein in its entirety.

As further illustrated in FIG. 1, the portable MRI device 100 may also include a conveyance mechanism 116 that allows the portable MRI device 100 to be transported to different locations. The conveyance mechanism 116 may include one or more components configured to facilitate movement of the portable MRI device 100, for example, to a location at which MRI may be needed. According to some embodiments, conveyance mechanism 116 may include a motor 118 coupled to drive wheels 120. In this manner, the conveyance mechanism 116 provides motorized assistance in transporting the MRI device 100 to desired locations. Additionally, the conveyance mechanism 116 may also include a plurality of casters 122 to assist with support and stability as well as facilitating transport.

In some embodiments, the conveyance mechanism 116 may optionally include motorized assistance controlled via a joystick (not shown) to guide the portable MRI device 100 during transportation to desired locations. According to some embodiments, the conveyance mechanism 116 may also include a power assist mechanism configured to detect when force is applied to the MRI device and, in response, to engage the conveyance mechanism 116 to provide motorized assistance in the direction of the detected force. For example, handles 124 may be configured to detect when force is applied thereto the rail (e.g., by personnel pushing on the handles 124) and engage the conveyance mechanism 116 to provide motorized assistance to drive the wheels 120 in the direction of the applied force. As a result, a user can guide the portable MRI device 100 with the assistance of the conveyance mechanism 116 that responds to the direction of force applied by the user.

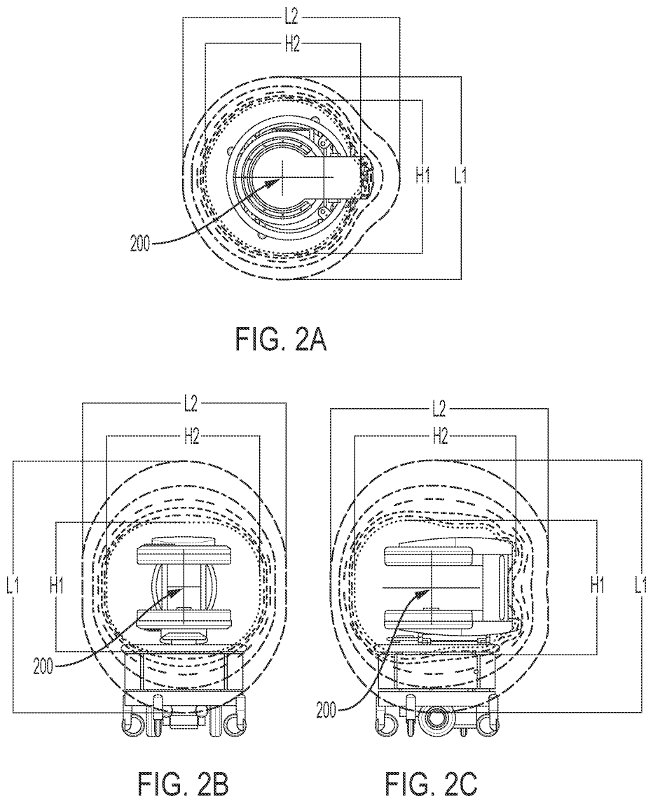

As indicated above, although the portable MRI device 100 operates at a B₀ field strength well below that of a traditional high-field system, there still may be concerns with access control, given certain fringe field strengths around an isocenter 200 of the B₀ magnet 104. By way of illustration, FIG. 2A, 2B and 2C are top, front and side views, respectively of a portable medical imaging device, for example, the device shown in FIG. 1. For example, an innermost region (defined by dimensions H1 and H2) may represent a 30 Gauss region and an outermost region (defined by dimensions L1 and L2) may represent a 5 Gauss region, wherein the fringe field strength decreases with increasing distance from the isocenter 200. Thus, one consideration in this regard may be, for example, the International Electrotechnical Commission (IEC) 60601-2-1933 standard, which defines controlled access as an area to which access may be controlled for safety reasons. The standard further specifies that a controlled access area around the MR equipment shall be defined such that outside this area: 1) the magnetic fringe field strength shall not exceed 0.5 mT and 2) the electromagnetic interference level complies with IEC 60601-1-2.

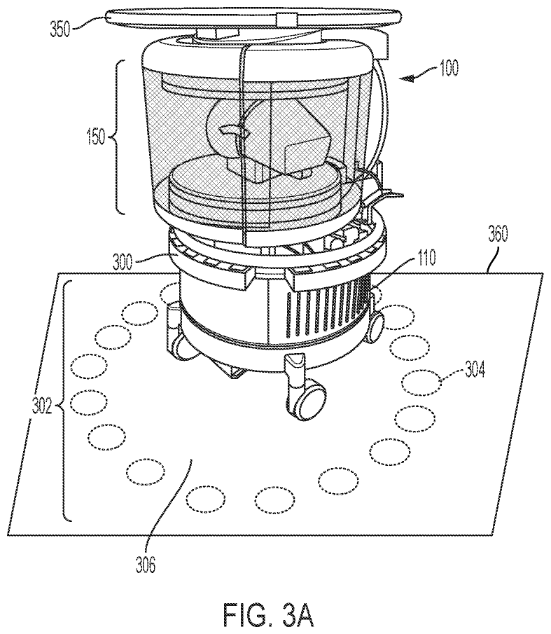

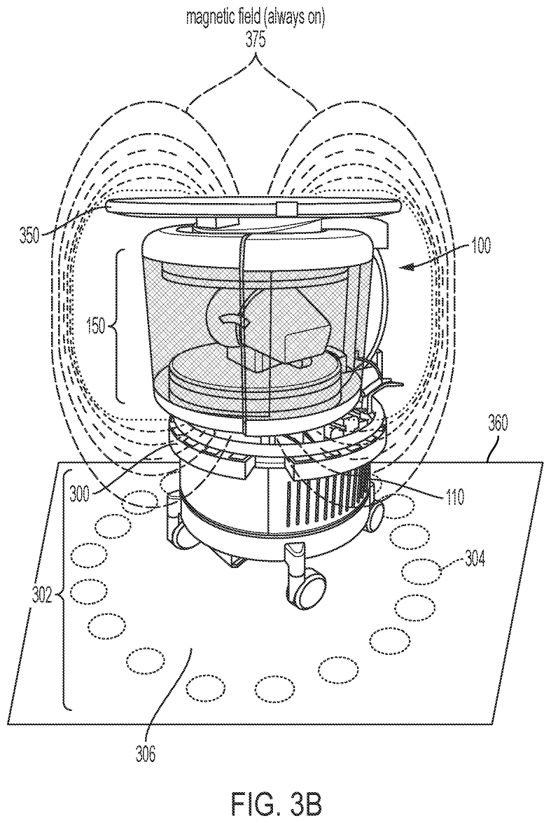

Accordingly, FIG. 3A illustrates the portable medical imaging device 100 of FIG. 1 with a device 300 configured to project a visible boundary around at least a portion of the portable medical imaging device, in accordance with some embodiments of the technology described herein. The device 300 may also be referred to herein as a guard, a Gauss guard, or a light Gauss guard.

The device 300 may be coupled to the MRI device 100. In the illustrated embodiment, the device 300 is shown being coupled to a base 110 of the MRI device 100 and below an imaging region 150 of the MRI device 100. In some embodiments, the device 300 may be coupled to the MRI device above the imaging region 150. In some embodiments, there may be multiple devices of the same type as device 300 being coupled to the MRI device 100 (e.g., above the imaging region 150 and below the imaging region 150).

As described herein, the device 300 may be configured to project a visible boundary 302 around at least a portion of the MRI device 100. In some embodiments, as shown in the illustrated embodiment, the visible boundary 302 surrounds the MRI device 100. In the illustrated example, the visible boundary 302 includes a collection of projections 304 from respective light sources. However, it should be appreciated that the visible boundary 302 may have any suitable shape and may be generated by any suitable light source or combination of light sources.

The visible boundary 302 may demarcate a region (e.g., inner region 306 shown in FIG. 3A) within which a magnetic field strength of a magnetic field generated by the MRI device 100 to which the device 300 is coupled equals or exceeds a threshold. For example, in some embodiments, the visible boundary corresponds to the 5 Gauss line for the MRI device 100. In some embodiments, a magnetic field strength within the inner region 306 may be between 1 Gauss and 30 Gauss. In some embodiments, a magnetic field strength within the inner region 306 may be between 5 Gauss and 20 Gauss. In some embodiments, a magnetic field strength within the inner region may be between 1 Gauss and 10 Gauss. In some embodiments, a magnetic field strength within the inner region may be greater than 30 Gauss.

As shown in FIG. 3A, the at least one light source may be arranged to, when operated, project the visible boundary onto a surface 360 which supports the portable MRI device (e.g., a ramp, a vehicle bed, and/or a floor).

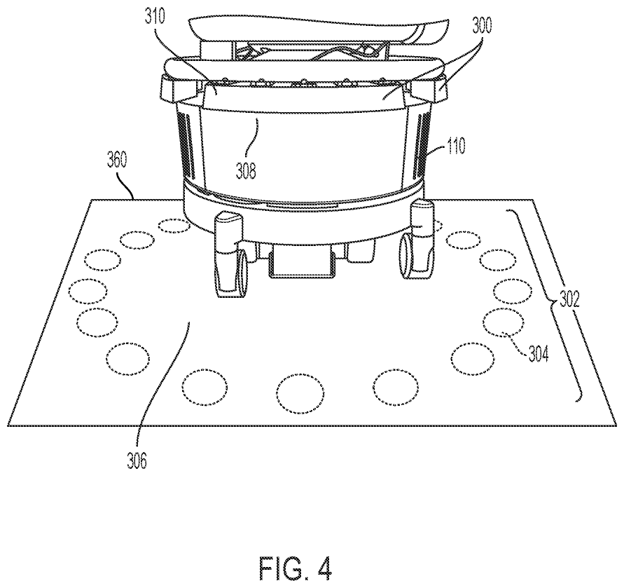

As shown in FIG. 4, for example, the device 100 may include at least one light source 308 arranged to, when operated, project the visible boundary 302 around at least a portion of the MRI device 100. In some embodiments, the at least one light source 308 comprises a plurality of light sources (e.g., at least 10 light sources, at least 20 light sources, any number of sources between 1 and 100, etc.). The light sources 308 may be arranged in a housing 310 of the device 300. The housing 310 may be coupled to the MRI device 100, as described herein.

In some embodiments, the light sources 308 may be arranged to encompass the MRI device 100 (e.g., being disposed around a perimeter of the MRI device 100). In some embodiments, the light sources 308 may be arranged around only a portion of the MRI device 100. The light sources 308 may be spaced equidistantly from each other.

Each of the light sources may be arranged to project a respective projection 304 around the MRI device 304. As shown in the illustrated embodiment, the visible boundary 302 comprises a plurality of projections 304. The projections 304 may be circular, in some embodiments. In other embodiments, any of the projections 304 may have any suitable shape. In some embodiments, all the projections 304 may have the same shape. In some embodiments, at least two of the projections 304 may have different shapes. In some embodiments, the projections may be spaced equidistantly from each other.

In some embodiments, the light sources may be adjustable. That is, an angle of a light source relative to the MRI device 100 (and/or to the housing 310) may be adjustable. Adjusting the angle of the light source relative to the MRI device 100 may change where light from the light source 308 is projected relative to the MRI device 100. Accordingly, a size and/or shape of the visible boundary 302 may be changed by adjusting the relative angle of one or more of the light sources 308 of the device. In some embodiments, the device 300 may be configured (e.g., with components such as the housing 310 and/or one or more fasteners) to hold the light sources 308 at fixed angles relative to the MRI device 100.

In some embodiments, the light sources 308 may be arranged to project a visible boundary that may be substantially symmetrical (e.g., radially symmetrical, circular, as in the illustrated embodiment). In some embodiments, the light sources 308 may be arranged to project a visible boundary that may be asymmetrical. The size and/or shape of the visible boundary may depend on the shape of the magnetic field and the strength of the magnetic field at various distances from the MRI device.

As described herein, the device may comprise one or more light sources arranged to project a visible boundary 302 on the surface 360. The visible boundary 302 may surround all or a portion of the MRI device 100. It should be appreciated that the visible boundary 302 may comprise any desired shape (e.g., circular, ovoidal, rectangular, etc.) and/or size. As described herein, in some embodiments, the visible boundary comprises text. In some embodiments, the visible boundary comprises a continuous boundary of light. In some embodiments, the visible boundary comprises a plurality of projections. In some embodiments, the visible boundary may comprise a picture or symbol, such as a warning symbol. In some embodiments, the one or more light sources may project one or more gobos on the surface 360. The gobo may be placed inside or in front of the one or more light sources to facilitate projection of a symbol, shape, or other image on the surface 360. Any suitable gobo may be implemented.

As described herein, the visible boundary 302 may correspond to a Gauss line for the MRI device 100 (e.g., a 5 Gauss line). In some embodiments, the visible boundary may illuminate a portion of surface 360 that extends beyond the Gauss line associated with the MRI device. In some embodiments, all or a portion of the inner region 306 may be illuminated by the one or more light sources of the device 300.

In some embodiments, operating the device 300 comprises providing power to the at least one light source 308 so that the light source 308 projects the visible boundary 302. In some embodiments, the light source 308 may be arranged to flash the visible boundary 302 (e.g., by alternating between projecting and not projecting the visible boundary 302). The device 300 may be configured to flash at a predefined frequency.

One or more additional alerts to draw nearby individuals' attention to the visible boundary 302 may be employed in combination with the device 300. For example, sounds, flashing patterns, and/or different colors may be implemented with the device. In some embodiments, one or more mechanical guards (e.g., mechanical guard 350 shown in FIG. 3A) may be employed with the device 300 to provide a physical barrier to the MRI device. For example, any of the guard devices described in U.S. Patent Publication No. US 2019/0324098, titled “DEPLOYABLE GUARD DEVICE FOR PORTABLE MAGNETIC RESONANCE IMAGING DEVICES”, filed Apr. 19, 2019, under Attorney Docket No.: O0354.70026US01, which is incorporated by reference herein in its entirety, may be implemented in combination with the device.

In some embodiments, a brightness of the light source 308 may be adjusted based on ambient lighting in an environment of the MRI device 100. For example, in some embodiments, the brightness of the light source 308 may be adjusted based on a brightness of the ambient lighting in the environment of the MRI device 100.

FIG. 3B illustrates the portable medical imaging device of FIG. 1 with a device configured to project a visible boundary 302 around at least a portion of the portable medical imaging device as well as the magnetic field 375 generated by the portable medical imaging device. In the example embodiment shown in FIG. 3B, the magnetic field 375 may be generated by one or more permanent magnets. As a result, the magnetic field 375 is always on—it is present because, unlike electromagnets, permanent magnets cannot be turned off.

In the example embodiment of FIG. 3B, the visible boundary 302 (the “light guard” in this example) may be turned on and off in any suitable way. For example, in some embodiments, the visible boundary 302 may be turned on (e.g., manually, for example, using a switch, a button, a computer command, or in any other suitable way) and stay on until turned off (e.g., also manually). In such a scenario the visible boundary 302 may be thought of as being “always on”.

As another example, in some embodiments, the visible boundary 302 may be turned automatically. For example, visible boundary 302 may be turned on automatically in response to being triggered by a sensor, for example, a sensor that detects when someone or something moves within a threshold distance of the portable medical imaging device. For example, in response to detecting, by a sensor, that a person or equipment has moved within a threshold distance of the portable medical imaging device, the visible boundary 302 may be turned on. In response to detecting, by the sensor, that the person or the equipment has moved outside of the threshold distance (which may also be detected by the sensor) the visible boundary 302 may be turned off.

In some embodiments, only a part of the visible boundary 302 may be turned on. For example, in some embodiments, only the light source(s) in the proximity of a person or equipment may be turned on. For example, when a sensor detects that a person has come within a threshold distance of the portable medical device, only a portion of the visible boundary 302 lights up—the portion to which the person would be closest.

In some embodiments, the visible boundary 302 may be turned on and may have a particular color and shape. In response to a sensor, detecting that a person or equipment is moved within a threshold distance of the portable medical imaging device, the color and/or shape of the visible boundary 302 may be changed. For example, the color of the visible boundary 302 may be green when no one is near the portable medical imaging device, but may be changed to red when a person moves within a threshold distance of the portable medical imaging device (e.g., as detected by a sensor). As another example, the shape of the visible boundary 302 may be changed to be larger than before, when a person moves within a threshold distance of the portable medical imaging device. As yet another example, the visible boundary 302 may project text (e.g., “stop”) when a person moves within a threshold distance of the portable medical imaging device.

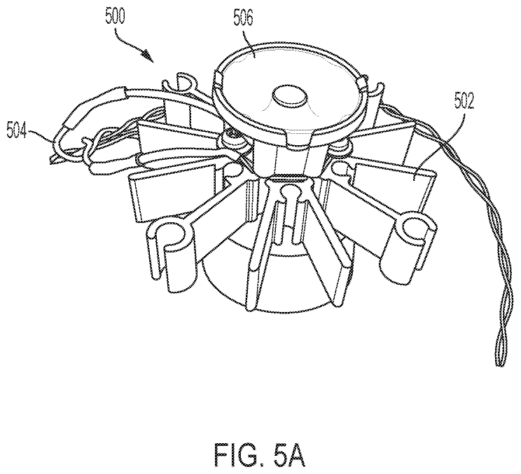

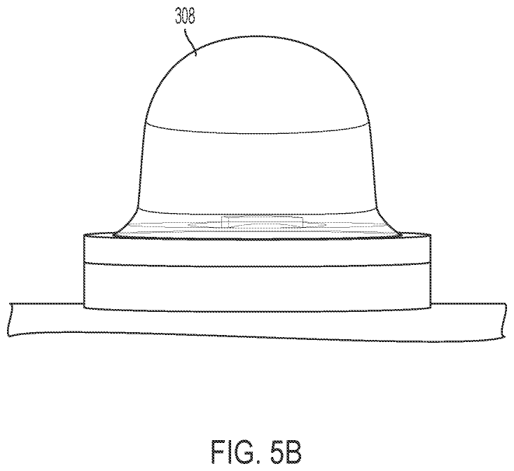

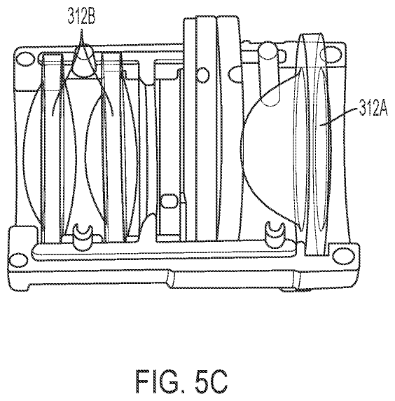

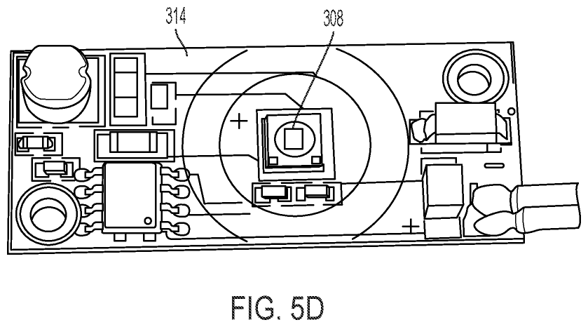

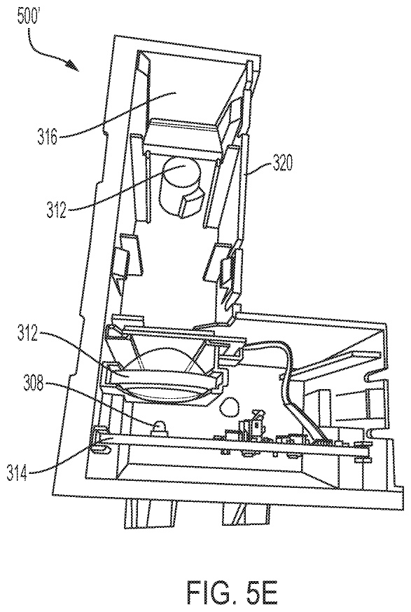

FIG. 5A illustrates an example optical module for device of FIG. 3A, in accordance with some embodiments of the technology described herein. As described herein, the device 300 may comprise one or more light sources 308. Each light source 308 may form part of an optical module 500, as shown in FIG. 5A. For example, the device 300 may comprise an optical module 500 and corresponding components for each light source 308. Accordingly, the device 300 may comprise a plurality of optical modules 500.

The optical module 500 may comprise a light source 308. In the illustrated embodiment, the optical module 500 further comprises a heat sink 502, and a power connection 504 (e.g., for providing power to the light source 308 to cause the light source to project the visible boundary 302), and TIR (total internal reflection) optic 506.

In some embodiments, the light source 308 described herein comprises a light emitting diode (LED). For example, FIG. 5B illustrates an example where the light source 308 used in the device comprises an LED.

In some embodiments, the light source may comprise a laser. Optical elements such as a Calle beam expander, beam homogenizer, diffuser, beam shaper, a rotating mirror, an oscillating mirror, or a combination thereof may be implemented in combination with the laser.

FIGS. 5C-5E illustrate further example components of an optical module 500 for the device of FIG. 3A, in accordance with some embodiments of the technology described herein. For example, as shown in FIG. 5C, the optical module 500 may comprise one or more lenses 312A, 312B. In the illustrated embodiment of FIG. 5C, one collimating lens 312A and two focusing lenses 312B are implemented. A light source may be placed to the right of aspherical lens 312A such that lens 312A collects light from the light source while lenses 312B focus the light. In some embodiments, a total internal reflection lens may be implemented in the optical module 500.

As shown in FIG. 5D, each optical module 500 may comprise a circuit board 314. In some embodiments, the circuit board 314 comprises components such as the heat sink and the light source 308. The circuit board 314 may facilitate controlling operation of the light source 308 to project the visible boundary 302.

FIG. 5E illustrates components of an example optical module 500′. As shown in FIG. 5E, the optical module 500 comprises a circuit board 314 having a light source 308, lenses 312, and a mirror 316. Components of the optical module 500′ may be disposed in optical module housing 320.

Although some specific embodiments of optical modules have been described herein, it should be appreciated that the optical module may comprise any combination of optical components. For example, any number and/or type of lenses, mirrors, or other optical elements may be implemented. Any type of lens may be used including Fresnel lenses, lens microarrays, TIR lenses, collimating lenses, etc.

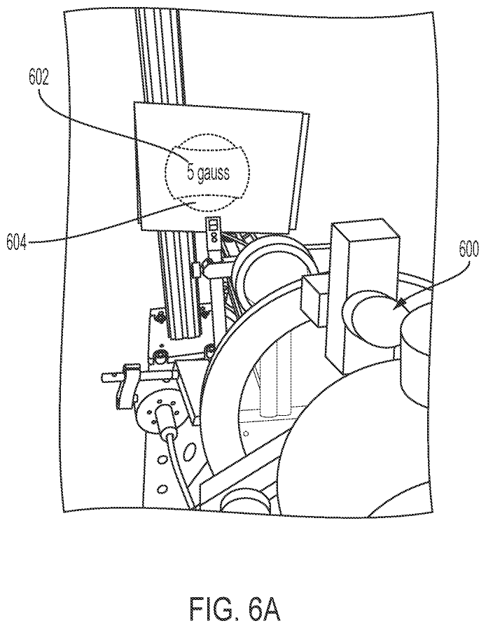

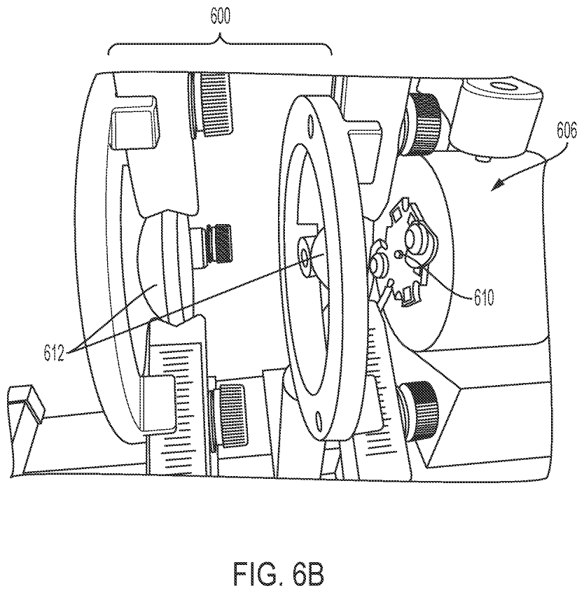

FIGS. 6A-6B illustrate another example optical module 600 for the device of FIG. 3A, in accordance with some embodiments of the technology described herein. In some embodiments, light sources of the device may be arranged to project a visible boundary that comprises text. For example, as shown in FIG. 6A, optical module 600 projects text 602 and a light projection 604. It should be appreciated that in some embodiments, the optical module projects only text 602 or only a light projection 604. The projected text 602 may comprise an indication of the magnetic field strength within the inner region, as described herein (e.g., an indication of the Gauss line where the text is projected such as “5 Gauss” as shown in the illustrated embodiment).

FIG. 6B illustrates example components of optical module 600 including light source 610 and lens 612. Light source 610 may be generally of the same type as light source 308 described herein. Optical module 600 may further comprise heatsink 606.

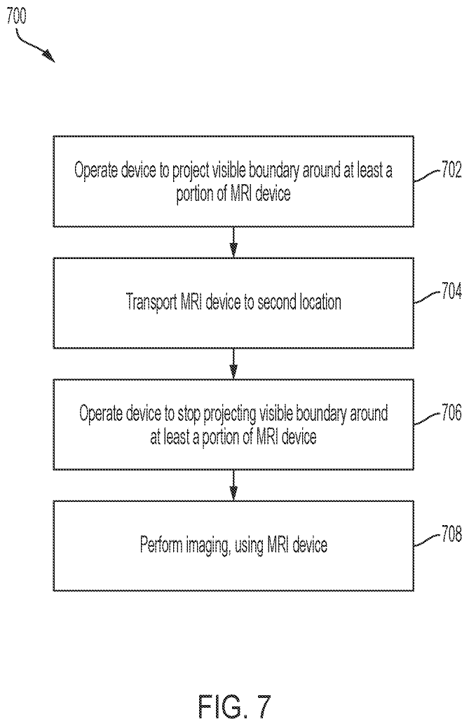

FIG. 7 illustrates an example process 700 for using device of FIG. 3A with a portable medical imaging device, in accordance with some embodiments of the technology described herein. Example process 700 may begin at act 702 wherein the device is operated to project a visible boundary around at least a portion of an MRI device.

The MRI device may optionally be transported 704 to a second location. In some embodiments, the MRI device is transported to the second location prior to imaging and/or while the device projects the visible boundary.

The device may optionally be operated 706 to stop projecting the visible boundary around the at least the portion of the MRI device.

Imaging 708 using the MRI device may be performed. In some embodiments, the visible boundary may be projected to demarcate a region having a defined magnetic field strength greater than or equal to a threshold while the MRI device is in operation to inhibit encroachment on the MRI device. In other embodiments, the visible boundary may be removed prior to performing imaging.

The technology described herein may be embodied in any of the following configurations:
(1) A device configured to be coupled to a portable magnetic resonance imaging (MRI) device, the device comprising: at least one light source arranged to, when operated, project a visible boundary around at least a portion of the portable MRI device, wherein the visible boundary demarcates a region within which a magnetic field strength of a magnetic field generated by the portable MRI device equals or exceeds a threshold.
(2) The device of (1), wherein the at least one light source is arranged to, when operated, project a visible boundary that surrounds the portable MRI device.
(3) The device of (1) or (2), wherein the at least one light source comprises a plurality of light sources.
(4) The device of any one of (1) through (3), wherein the at least one light source comprises a first light source, wherein an angle of the first light source relative to the portable MRI device is adjustable such that adjusting the angle of the first light source changes a shape and/or size of the visible boundary.
(5) The device of any one of (1) through (4), wherein the at least one light source comprises multiple light sources, and wherein respective angles of the multiple light sources may be adjusted independently of one another such that different light sources of the multiple light sources may be positioned at different angles relative to the portable MRI device.
(6) The device of (4), wherein the device is further configured to hold the first light source at a fixed angle relative to the portable MRI device.
(7) The device of any one of (1) through (6), wherein the at least one light source is arranged to, when operated, project a visible boundary comprising text.
(8) The device of (7), wherein the text comprises an indication of the magnetic field strength at or within the visible boundary.
(9) The device of any one of (1) through (8), wherein a brightness of the at least one light source is set based on a brightness of ambient lighting in an environment of the portable MRI device.
(10) The device of any one of (1) through (9), wherein the magnetic field strength within the region is between 1 Gauss and 30 Gauss.
(11) The device of any one of (1) through (10), wherein the magnetic field strength within the region is between 1 Gauss and 10 Gauss.
(12) The device of any one of (1) through (11), wherein the visible boundary indicates a 5 Gauss line of the portable MRI device.
(13) The device of any one of (1) through (12), wherein the at least one light source is arranged to, when operated, project a plurality of projections.
(14) The device of (13), wherein respective ones of the plurality of projections are spaced equidistantly from each other.
(15) The device of any one of (1) through (14), wherein the at least one light source is arranged to, when operated, project a visible boundary that is radially symmetrical.
(16) The device of any one of (1) through (15), wherein the at least one light source is arranged to, when operated, project a visible boundary that is asymmetrical.
(17) The device of any one of (1) through (16), wherein the at least one light source is arranged to, when operated, project a continuous projection of light.
(18) The device of any one of (1) through (17), wherein the at least one light source is arranged to, when operated, alternate between projecting the visible boundary and not projecting the visible boundary.
(19) The device of (18), wherein the at least one light source alternates between projecting the visible boundary and not projecting the visible boundary at a predefined frequency.
(20) The device of any one of (1) through (19), wherein the at least one light source is arranged to, when operated, project the visible boundary onto a surface which supports the portable MRI device.
(21) The device of (20), wherein the surface comprises a ramp.
(22) The device of (20), wherein the surface comprises a vehicle bed.
(23) The device of (20), wherein the surface comprises a floor.
(24) A system, comprising: a portable magnetic resonance imaging (MRI) device; and a device coupled to the portable MRI device comprising at least one light source arranged to, when operated, project a visible boundary around at least a portion of the portable MRI device, wherein the visible boundary demarcates a region within which magnetic field strength of a magnetic field generated by the portable MRI device equals or exceeds a threshold.
(25) The system of (24), wherein the device is coupled to the portable MRI device below an imaging region of the portable MRI device.
(26) The system of (24) or (25), wherein the device is coupled to the portable MRI device above an imaging region of the portable MRI device.
(27) The system of any one of (24) through (26), wherein the portable MRI device further comprises a base, the base supporting a magnetics system of the portable MRI device and housing a power system for the portable MRI device, wherein the base further comprises at least one conveyance mechanism allowing the portable MRI device to be transported to different locations, and wherein the device is coupled to the base of the portable MRI device.
(28) The system of any one of (24) through (27), wherein the at least one light source is arranged to, when operated, project a visible boundary that surrounds the portable MRI device.
(29) The system of any one of (24) through (28), wherein the at least one light source comprises a plurality of light sources.
(30) The system of any one of (24) through (29), wherein the at least one light source comprises a first light source, wherein an angle of the first light source relative to the portable MRI device is adjustable such that adjusting the angle of the first light source changes a size and/or shape of the visible boundary.
(31) The system of (30), wherein the at least one light source comprises multiple light sources, and wherein respective angles of the multiple light sources may be adjusted independently of one another such that different light sources of the multiple light sources may be positioned at different angles relative to the portable MRI device.
(32) The system of (30), wherein the device is further configured to hold the first light source at a fixed angle relative to the portable MRI device.
(33) The system of any one of (24) through (32), wherein the at least one light source is arranged to, when operated, project a visible boundary comprising text.
(34) The system of (33), wherein the text comprises an indication of the magnetic field strength of the visible boundary.
(35) The system of any one of (24) through (34), wherein a brightness of the at least one light source is set based on a brightness of ambient lighting in an environment of the portable MRI device.
(36) The system of any one of (24) through (35), wherein the magnetic field strength within the region is between 1 Gauss and 30 Gauss.
(37) The system of any one of (24) through (36), wherein the magnetic field strength within the region is between 1 Gauss and 10 Gauss.
(38) The system of any one of (24) through (37), wherein the visible boundary indicates a 5 Gauss line of the portable MRI device.
(39) The system of any one of (24) through (38), wherein the at least one light source is arranged to, when operated, project a plurality of projections.
(40) The system of (39), wherein respective ones of the plurality of projections are spaced equidistantly from each other.
(41) The system of any one of (24) through (40), wherein the at least one light source is arranged to, when operated, project a visible boundary that is radially symmetrical.
(42) The system of any one of (24) through (41), wherein the at least one light source is arranged to, when operated, project a visible boundary that is asymmetrical.
(43) The system of any one of (24) through (42), wherein the at least one light source is arranged to, when operated, project a continuous projection of light.
(44) The system of any one of (24) through (43), wherein the at least one light source is arranged to, when operated, alternate between projecting the visible boundary and not projecting the visible boundary.
(45) The system of (44), wherein the at least one light source alternates between projecting the visible boundary and not projecting the visible boundary at a predefined frequency.
(46) The system of any one of (24) through (45), wherein the at least one light source is arranged to, when operated, project the visible boundary onto a surface which supports the portable MRI device.
(47) The system of (46), wherein the surface comprises a ramp.
(48) The system of (46), wherein the surface comprises a vehicle bed.
(49) The system of (46), wherein the surface comprises a floor.
(50) A method for operating a magnetic resonance imaging (MRI) device, the MRI device being coupled to a device comprising at least one light source arranged to, when operated, project a visible boundary around at least a portion of the portable MRI device, wherein the visible boundary demarcates a region within which a magnetic field strength of a magnetic field generated by the portable MRI device equals or exceeds a threshold, the method comprising: operating the device to project the visible boundary; prior to imaging, using the MRI device, operating the device to stop projecting the visible boundary; and imaging, using the MRI device.
(51) The method of (50), further comprising, while operating the device to project the visible boundary, transporting the MRI device to a second location.
(52) The method of (50) or (51), wherein operating the device to project the visible boundary comprises powering the at least one light source so that the at least one light source projects the visible boundary around at least the portion of the MRI device.
(53) The method of any one of (50) through (52), wherein operating the device to project the visible boundary comprises operating the at least one light source to alternate between projecting the visible boundary and not projecting the visible boundary.

### Alternatives and Scope

The above-described embodiments can be implemented in any of numerous ways. For example, the embodiments may be implemented using hardware, software or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the technology described herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately,” “substantially,” and “about” may be used to mean within ±20% of a target value in some embodiments, within ±10% of a target value in some embodiments, within ±5% of a target value in some embodiments, and within ±2% of a target value in some embodiments. The terms “approximately” and “about” may include the target value.

What is claimed is:

1. A device configured to be coupled to a portable magnetic resonance imaging (MRI) device, the device comprising:
at least one light source arranged to, when operated, project a visible boundary around at least a portion of the portable MRI device, wherein the visible boundary demarcates a region within which a magnetic field strength of a magnetic field generated by the portable MRI device equals or exceeds a threshold.

2. The device of claim 1, wherein the at least one light source is arranged to, when operated, project a visible boundary that surrounds the portable MRI device.

3. The device of claim 1 or any other preceding claim, wherein the at least one light source comprises a plurality of light sources.

4. The device of claim 1 or any other preceding claim, wherein the at least one light source comprises a first light source, wherein an angle of the first light source relative to the portable MRI device is adjustable such that adjusting the angle of the first light source changes a shape and/or size of the visible boundary.

5. The device of claim 1 or any other preceding claim, wherein the at least one light source comprises multiple light sources, and wherein respective angles of the multiple light sources may be adjusted independently of one another such that different light sources of the multiple light sources may be positioned at different angles relative to the portable MRI device.

6. The device of claim 4, wherein the device is further configured to hold the first light source at a fixed angle relative to the portable MRI device.

7. The device of claim 1 or any other preceding claim, wherein the at least one light source is arranged to, when operated, project a visible boundary comprising text.

8. The device of claim 7, wherein the text comprises an indication of the magnetic field strength at or within the visible boundary.

9. The device of claim 1 or any other preceding claim, wherein a brightness of the at least one light source is set based on a brightness of ambient lighting in an environment of the portable MRI device.

10. The device of claim 1 or any other preceding claim, wherein the magnetic field strength within the region is between 1 Gauss and 30 Gauss.

11. The device of claim 1 or any other preceding claim, wherein the magnetic field strength within the region is between 1 Gauss and 10 Gauss.

12. The device of claim 1 or any other preceding claim, wherein the visible boundary indicates a 5 Gauss line of the portable MRI device.

13. The device of claim 1 or any other preceding claim, wherein the at least one light source is arranged to, when operated, project a plurality of projections.

14. The device of claim 13, wherein respective ones of the plurality of projections are spaced equidistantly from each other.

15. The device of claim 1 or any other preceding claim, wherein the at least one light source is arranged to, when operated, project a visible boundary that is radially symmetrical.

16. The device of claim 1 or any other preceding claim, wherein the at least one light source is arranged to, when operated, project a visible boundary that is asymmetrical.

17. The device of claim 1 or any other preceding claim, wherein the at least one light source is arranged to, when operated, project a continuous projection of light.

18. The device of claim 1 or any other preceding claim, wherein the at least one light source is arranged to, when operated, alternate between projecting the visible boundary and not projecting the visible boundary.

19. The device of claim 18, wherein the at least one light source alternates between projecting the visible boundary and not projecting the visible boundary at a predefined frequency.

20. The device of claim 1 or any other preceding claim, wherein the at least one light source is arranged to, when operated, project the visible boundary onto a surface which supports the portable MRI device.

21. The device of claim 20, wherein the surface comprises a ramp, a vehicle bed or a floor.

22. A system, comprising:
a portable magnetic resonance imaging (MRI) device; and
a device coupled to the portable MRI device comprising at least one light source arranged to, when operated, project a visible boundary around at least a portion of the portable MRI device, wherein the visible boundary demarcates a region within which a magnetic field strength of a magnetic field generated by the portable MRI device equals or exceeds a threshold.

23. A method for operating a magnetic resonance imaging (MRI) device, the MRI device being coupled to a device comprising at least one light source arranged to, when operated, project a visible boundary around at least a portion of the portable MRI device, wherein the visible boundary demarcates a region within which a magnetic field strength of a magnetic field generated by the portable MRI device equals or exceeds a threshold, the method comprising:
operating the device to project the visible boundary;
prior to imaging, using the MRI device, operating the device to stop projecting the visible boundary; and
imaging, using the MRI device.
